(19) United States
(12) Patent Application Publication
(10) Pub. No.: US 2005/0070473 A9
(43) Pub. Date: Mar. 31, 2005

(54) Methods of screening for apoptosis-controlling agents for bone anabolic therapies and uses thereof

(76) Inventors: Stavros C. Manolagas, Little Rock, AR (US); Robert L. Jilka, Little Rock, AR (US); Robert S. Weinstein, Little Rock, AR (US); Teresita Bellido, Little Rock, AR (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/743,360

(22) Filed: Dec. 22, 2003

Related U.S. Application Data
(63) Continuation of application No. 09/413,785, filed on Oct. 7, 1999, now abandoned.
(60) Provisional application No. 60/116,409, filed on Jan. 19, 1999.
(60) Provisional application No. 60/103,385, filed on Oct. 7, 1998.

(51) Int. Cl. A61K 38/29

(52) U.S. Cl. 514/12

(57) ABSTRACT

The present invention demonstrates that human parathyroid hormone 1-34 [hPTH(1-34)] exerts anti-apoptotic effects on osteoblasts when administered in an intermittent fashion to mice in vivo. The present invention further demonstrates that bovine PTH(1-34) [bPTH(1-34)] prevents glucocorticoid-induced apoptosis of osteoblastic and osteocytic cells in vitro. Therefore, the present invention demonstrates that the previously established anabolic effects of PTH on the skeleton are mediated by its ability to postpone osteoblast apoptosis, as opposed to a stimulatory effect on osteoblastogenesis. The present invention provides methods of screening agents for anti-apoptotic effects on osteoblasts, wherein such agents stimulate and/or restore bone in osteopenic individuals, or prevent bone loss caused by agents such as glucocorticoids.

### RELATED APPLICATIONS

This application is a continuation of U.S. application Ser. No. 09/413,785, filed Oct. 7, 1999, which claims the benefit of U.S. Provisional Application No. 60/116,409, filed Jan. 19, 1999 and U.S. Provisional Application No. 60/103,385, filed Oct. 7, 1998. The entire teachings of the above application(s) are incorporated herein by reference.

### FEDERAL FUNDING LEGEND

This invention was produced in part using funds obtained through grant PO1-AG13918 from the National Institutes of Health. Consequently, the federal government has certain rights in this invention.

### BACKGROUND OF THE INVENTION

1. Field of the Invention

The present invention relates generally to bone physiology. More specifically, the present invention relates to inhibiting apoptosis of osteoblasts and osteocytes.

2. Description of the Related Art

Remodeling of the human adult skeleton is carried out by teams of juxtaposed osteoclasts and osteoblasts. Osteoclasts and osteoblasts are specialized cell types that originate from hematopoietic and mesenchymal progenitors of the bone marrow, respectively. During bone remodeling, old bone is resorbed by osteoclasts and replaced with new bone by osteoblasts. After they have completed bone matrix synthesis, osteoblasts become osteocytes or lining cells, or they undergo apoptosis.

The osteoblasts and osteoclasts that carry out bone remodeling comprise the basic multi-cellular unit (BMU). Because the lifetime of the basic multi-cellular unit is longer than the lifetime of the individual osteoclasts and osteoblasts, new cells must be continuously supplied from progenitors in the bone marrow for progression to occur. Continuous and orderly supply of these cells, as well as the appropriate rate of apoptosis, is essential for bone homeostasis, as increased or decreased production of osteoclasts or osteoblasts leads to osteoporosis, Paget's, metastatic and renal bone disease. Little is known, however, about the factors that regulate osteogenesis in postnatal life and how osteoblastogenesis and osteoclastogenesis are coordinated to ensure a balance between formation and resorption during remodeling.

During the last few years, it has been established that the process of bone remodeling is regulated locally by growth factors and cytokines produced in the bone micro-environment. In addition, systemic hormones modulate the production and/or action of locally produced cytokines and growth factors, thereby influencing the rate of bone remodeling. Bone morphogenetic proteins (BMPs) are unique among growth factors that influence osteoblast differentiation because they can initiate this process from uncommitted progenitors in vitro as well as in vivo.

Osteoblast commitment is mediated by the type I bone morphogenetic proteins receptor and involves the phosphorylation of specific transactivators (smad 1, 5 and possibly 9), which then oligomerize with smad 4 and translocate into the nucleus. These events induce an osteoblast specific transcription factor (OSF-2/cbfa-1/PEBP2aA/AML3), which in turn activates osteoblast-specific genes (6,7). Bone morphogenetic protein-2 and bone morphogenetic protein-4 are expressed during murine embryonal skeletogenesis (day 10-12) and act on cells isolated from murine limb buds to promote their differentiation into osteoblasts. In addition, bone morphogenetic protein-2 and bone morphogenetic protein-4 are involved in fracture healing, as evidenced by their expression in primitive mesenchymal cells and chondrocytes at the site of callus formation, as well as the ability of bone morphogenetic proteins to accelerate the fracture healing process when supplied exogenously.

Bone morphogenetic proteins play an essential role in the differentiation of cells that provide support for osteoclast development. Osteoclast development requires support from stromal/osteoblastic cells. Moreover, in vivo, osteoclastogenesis and osteoblastogenesis proceed simultaneously in most circumstances. This dependency is mediated by a membrane bound cytokine-like molecule (osteoprotogerin ligand/RANK ligand) present in mesenchymal cells which binds to a specific receptor on osteoclast progenitor cells. Such binding is essential, and together with M-CSF, sufficient, for osteoclastogenesis.

The adverse effects of hypercortisolism on bone have been recognized for over 60 years, but the precise cellular and molecular basis of these changes has remained elusive. Today, the iatrogenic form of the disease has become far more common than Cushing's syndrome and glucocorticoid-induced osteoporosis is now third in frequency following post-menopausal and senile osteoporosis.

Bone loss due to glucocorticoid excess is diffuse, affecting both cortical and cancellous bone, but has a predilection for the axial skeleton. Spontaneous fractures of the vertebrae or ribs are, therefore, often presenting manifestations of the disorder. A cardinal feature of glucocorticoid-induced osteoporosis is decreased bone formation. In addition, patients receiving long-term glucocorticoid therapy sometimes develop collapse of the femoral head (osteonecrosis), but the mechanism underlying this is uncertain. Decreased bone formation, and in situ death of isolated segments of the proximal femur suggest that glucocorticoid excess may alter the birth and death of bone cells. Defective osteoblastogenesis has been reported to be linked to reduced bone formation and age-related osteopenia in the SAMP6 mouse. Besides the relationship between aberrant osteoblast production and osteoporosis, it has been recently shown that a significant proportion of osteoblasts undergo apoptosis, which raises the possibility that the premature or more frequent occurrence of osteoblast apoptosis could contribute to incomplete repair of resorption cavities and loss of bone.

Once osteoblasts have completed their bone-forming function, they either die by apoptosis, become entrapped in bone matrix and become osteocytes, or remain on the surface as lining cells. Previous studies have demonstrated that the number of osteoblasts is a critical determinant of bone formation, and that the osteopenic effects of glucocorticoids are due, at least in part, to acceleration of osteoblast apoptosis and stimulation of osteocyte apoptosis.

While several agents are capable of decreasing bone resorption and halting further bone loss in osteopenic states, the ideal drug would be an anabolic agent that increases bone mass by rebuilding bone. It is well established that daily injections of low doses of parathyroid hormone (PTH), an agent better known for its role in calcium homeostasis, increases bone mass in animals and humans (5) as does the PTH-related protein (PTHrP), the only other known ligand of the PTH receptor (6). The mechanism of these anabolic effects, however, has not been established.

The prior art is deficient in methods of inhibiting apoptosis of osteoblasts and osteocytes. The present invention fulfills this long-standing need and desire in the art.

### SUMMARY OF THE INVENTION

Intermittent PTH administration increases bone mass, but the mechanism of this effect has remained heretofore unknown. Daily PTH injections in mice either with normal bone mass or osteopenia due to defective osteoblastogenesis increased bone formation without affecting the generation of new osteoblasts. Instead, PTH did increase the life span of mature osteoblasts by preventing their apoptosis, an effect reproduced in vitro. Increasing the work performed by a cell population to augment tissue mass by suppressing apoptosis represents a novel biologic paradigm for regenerating tissues; and could provide a pharmacotherapeutic strategy for rebuilding bone in patients with established osteopenia.

Evidence is presented herein that human parathyroid hormone 1-34 [hPTH(1-34)] exerts anti-apoptotic effects on osteoblasts when administered in an intermittent fashion to mice in vivo. The present invention also provides evidence that bovine PTH(1-34) [bPTH(1-34)] prevents glucocorticoid-induced apoptosis of osteoblastic and osteocytic cells in vitro.

One object of the present invention is to provide methods for screening compounds that prevent osteoblast apoptosis, thereby stimulating bone formation and/or restoring bone in osteopenic individuals, or preventing bone loss caused by agents such as glucocorticoids.

In an embodiment of the present invention, there is provided a method of reducing the number of osteoblasts undergoing apoptosis in an individual in need of such treatment, comprising the step of: administering a therapeutic dose of human parathyroid hormone [hPTH(1-34)] to said individual, wherein administration of human parathyroid hormone [hPTH(1-34)] results in a reduction in the number of osteoblasts undergoing apoptosis, thereby reducing bone loss and/or stimulating bone formation in said individual.

In another embodiment of the present invention, there is provided a method of screening compounds that stimulate bone formation, comprising the steps of: (a) contacting osteoblast cells with a test compound; (b) determining the number of said cells undergoing apoptosis; and (c) comparing the number of apoptotic cells with osteoblast cells that have not been contacted with said compound, wherein fewer apoptotic cells following contact with said compound than in the absence of said contact indicates that said compound inhibits apoptosis resulting in stimulation of bone formation.

In yet another embodiment of the present invention, there is provided a method of screening for compounds that decrease bone loss, comprising the steps of: (a) treating osteoblast cells with a glucocorticoid; (b) contacting said osteoblast cells with a test compound; (c) determining the number of said osteoblast cells undergoing apoptosis; and (d) comparing the number of apoptotic cells with osteoblast cells that have been treated with said glucocorticoid but were not contacted with said test compound, wherein fewer apoptotic cells following contact with said test compound than in the absence of said contact with said test compound indicates that said compound inhibits apoptosis of osteoblast cells thereby reducing bone loss.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows glucocorticoid-induced apoptosis of osteoblastic cells inhibited by the specific caspase-3 inhibitor, DEVD.

FIG. 2 shows that parathyroid hormone blocks glucocorticoid-induced, but not TNFα-induced, apoptosis of osteoblastic cells.

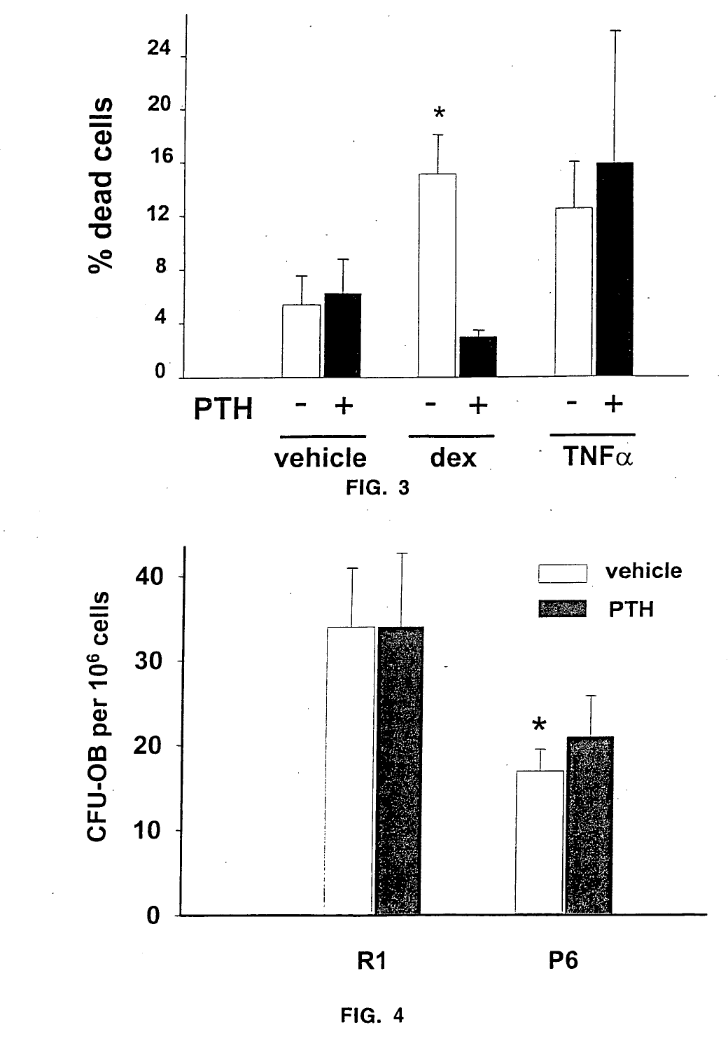

FIG. 3 shows that parathyroid hormone blocks glucocorticoid-induced, but not TNFα-induced, apoptosis of MLO-Y4 osteocytes.

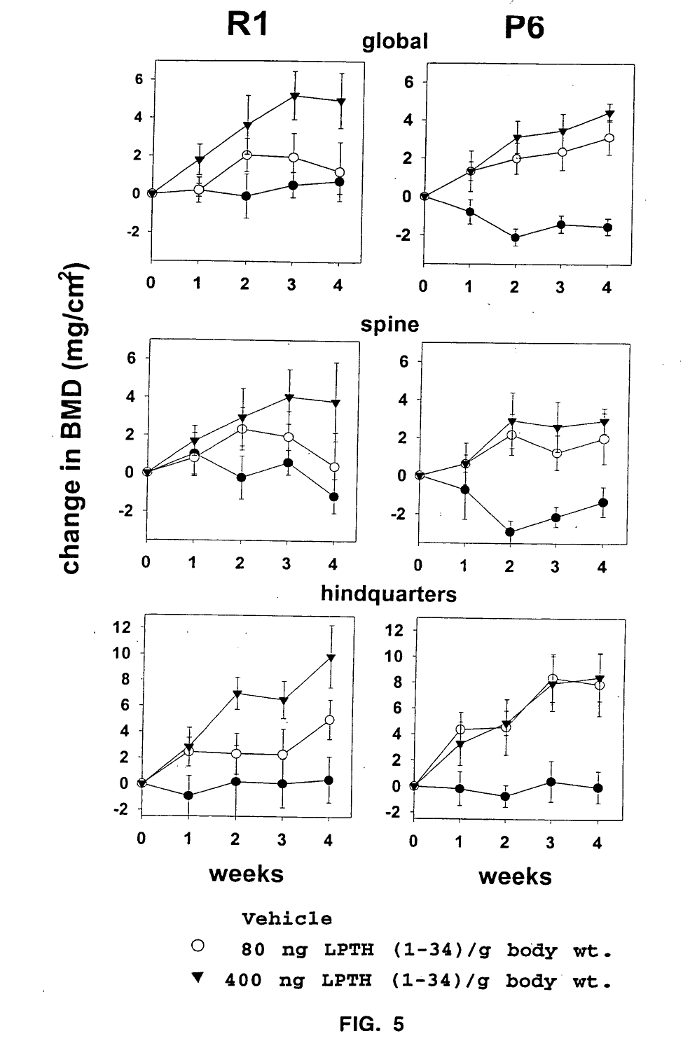

FIG. 4 shows that PTH fails to stimulate osteoblastogenesis.

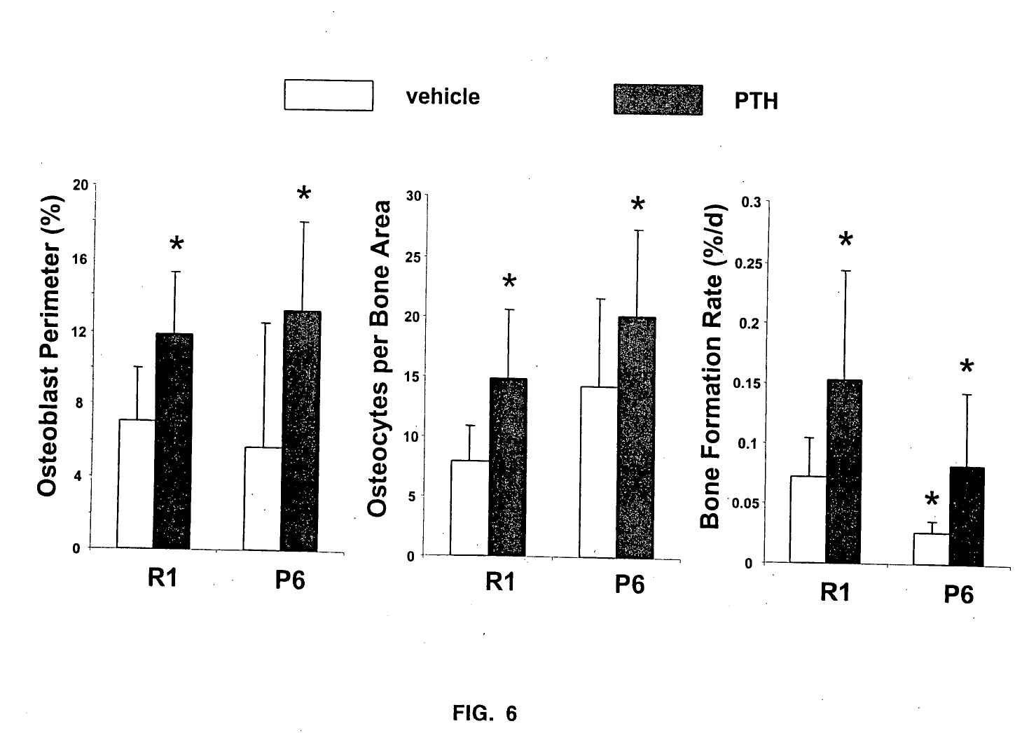

FIG. 5 shows the BMD changes in PTH-treated mice.

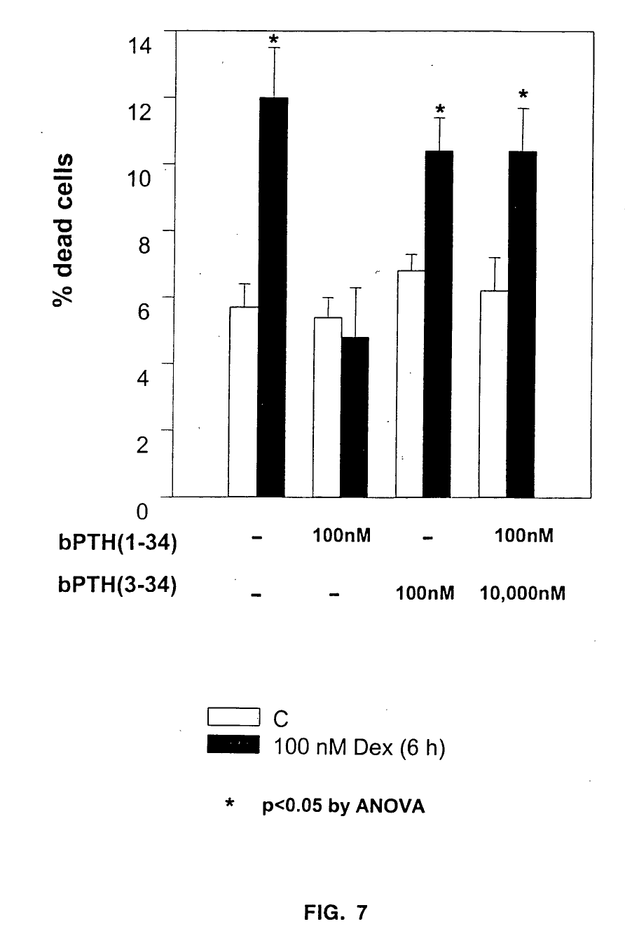

FIG. 6 shows that PTH stimulates osteoblast and osteocyte number as well as bone formation rate.

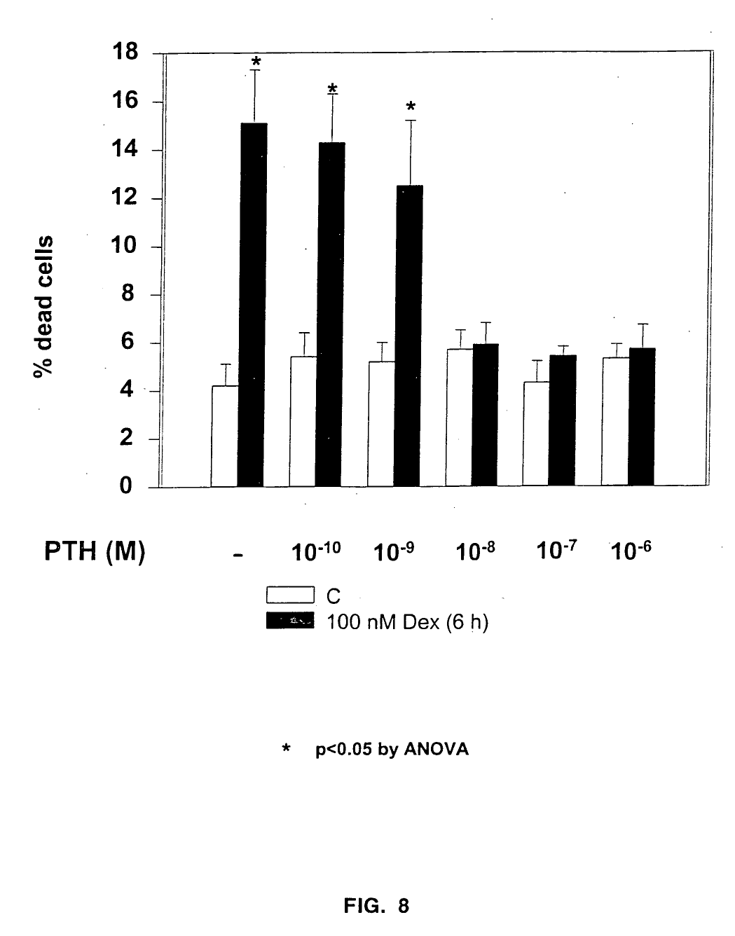

FIG. 7 shows that bPTH(1-34) blocks glucocorticoid-induced apoptosis and bPTH(3-34) prevents the anti-apoptotic effect of 1-34 PTH.

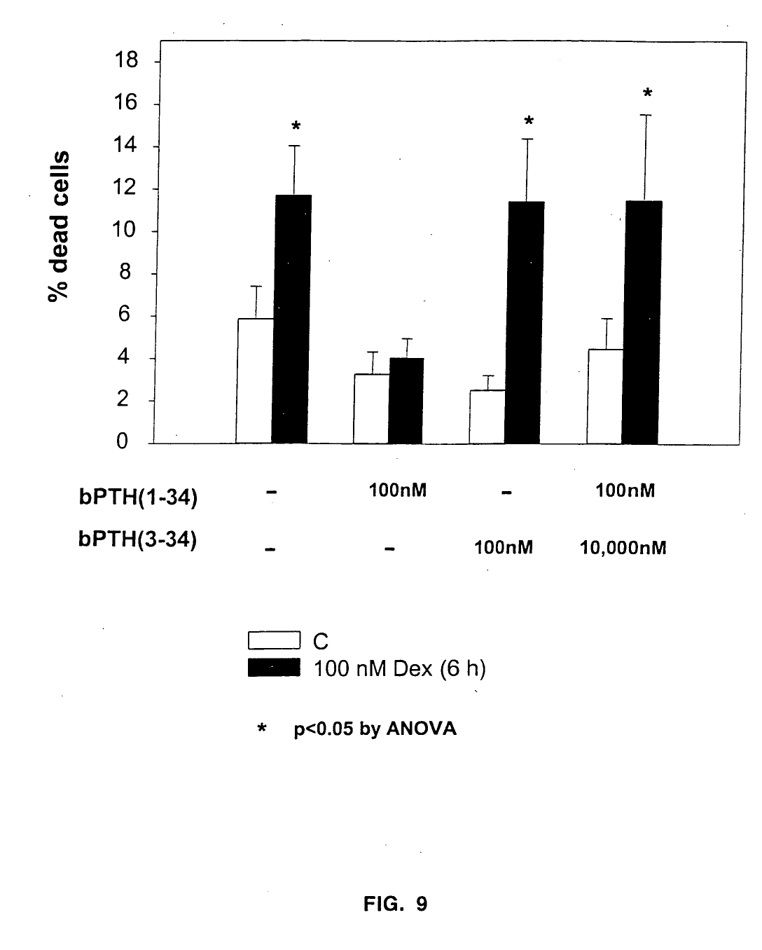

FIG. 8 shows that PTH blocks glucocorticoid-induced apoptosis of osteoblastic cells.

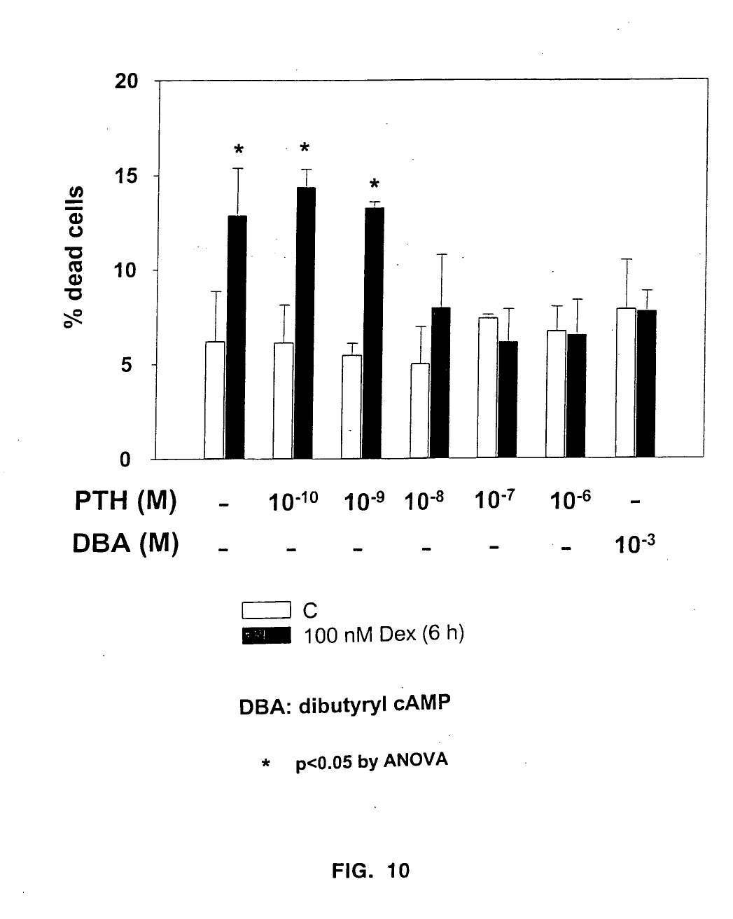

FIG. 9 shows that bPTH(1-34) blocks glucocorticoid-induced apoptosis of MLO-Y4 osteocytes and bPTH(3-34) prevents the anti-apoptotic effect of 1-34 PTH.

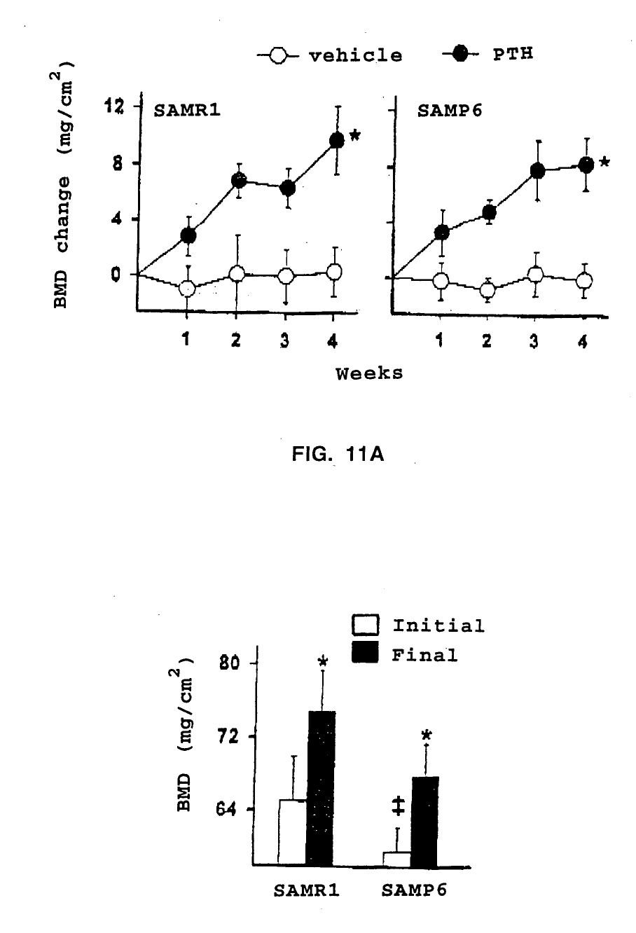

FIG. 10 shows that PTH and the cAMP analog, DBA, block glucocorticoid-induced apoptosis of MLO-Y4 osteocytes.

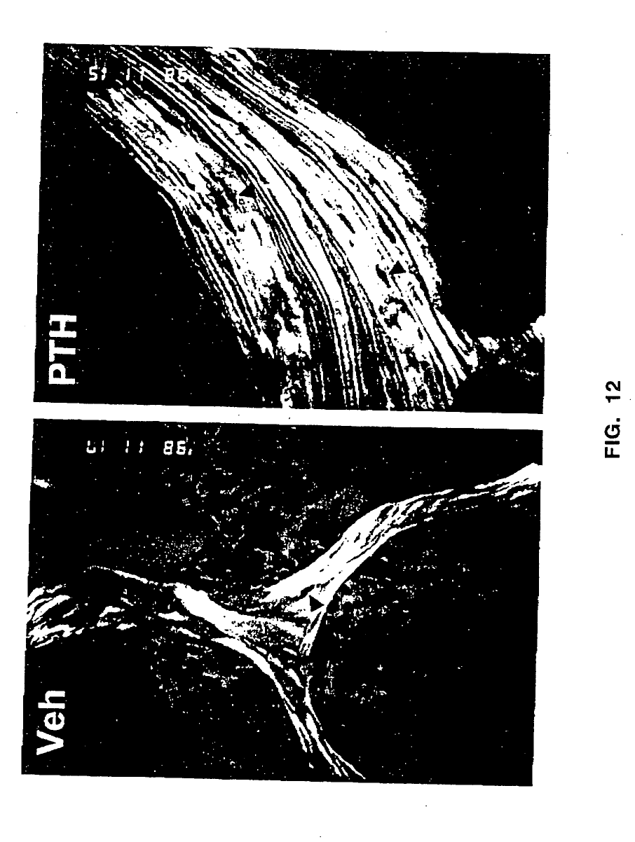

FIG. 11 shows the effect of PTH on BMD. FIG. 11A, Each point represents the mean (±s.d.) change in hindlimb BMD from base line. * P<0.05 vs. vehicle established using a mixed effects longitudinal ANOVA model (Procmixed, SAS, Cary, N.C.) to allow specification of the covariance structure. FIG. 11B, Mean (±s.d.) BMD of hindlimb of SAMR1 and SAMP6 mice prior to (“initial”) and after (“final”) 28 days of treatment with hPTH(1-34). * P<0.05 vs. initial by paired t-test; P<0.05 vs. SAMR1 by Studentís t-test.

FIG. 12 shows distal femoral cancellous bone viewed with polarized light to reveal lamellar architecture. Arrows indicate osteocytes. Original magnification=200×.

FIG. 13 shows the mechanism and signal specificity of the suppressive effect of PTH on apoptosis in cultures of osteoblastic and osteocytic cells. FIG. 13A, Inhibition of dexamethasone-induced apoptosis of calvaria cells and MLO-Y4 cells by PTH. Original magnification 400×. Insets: % of cells undergoing apoptosis determined from evaluation of 200 cells in randomly selected fields. FIG. 13B, Cells (10⁴ per cm²) were incubated for 1 hour in vehicle (Veh) or 10⁻⁸ M bPTH(1-34), and then for an additional 6 hours in the absence (“basal”) or presence of 5×10⁻⁵ M etoposide (“etop”), 10⁻⁷ M dexamethasone (“dex”), or 10⁻⁹ M TNF. FIG. 13C, Osteoblastic calvaria cells were cultured for 1 hour in vehicle or the indicated log molar concentrations of bPTH(1-34), bPTH(3-34) or DB-cAMP, and then for an additional 6 hours in the absence or presence of 10⁻⁷ M dexamethasone. Adherent cells were released by digestion with trypsin-EDTA, combined with nonadherent cells, and apoptotic cells enumerated by trypan blue staining (7). Bars represent the mean (±s.d.) of 3 independent measurements. Cell death induced by etoposide, dexamethasone and TNF was blocked by DEVD-CHO, a cell permeable inhibitor of caspases required for the execution phase of apoptosis (21). Data were analyzed by ANOVA. Etoposide, dexamethasone, and TNF caused a significant (p<0.05) increase in apoptosis in cultures containing vehicle. * p<0.05 vs. vehicle (A), or vs. dexamethasone alone (B).

### DETAILED DESCRIPTION OF THE INVENTION

Intermittent PTH administration increases bone mass, but the mechanism of this effect has remained heretofore unknown. Daily PTH injections in mice either with normal bone mass or osteopenia due to defective osteoblastogenesis increased bone formation without affecting the generation of new osteoblasts. Instead, PTH did increase the life span of mature osteoblasts by preventing their apoptosis, an effect reproduced in vitro. Increasing the work performed by a cell population to augment tissue mass by suppressing apoptosis represents a novel biologic paradigm for regenerating tissues; and could provide a pharmacotherapeutic strategy for rebuilding bone in patients with established osteopenia.

Evidence is presented herein that human parathyroid hormone 1-34 [hPTH(1-34)] exerts anti-apoptotic effects on osteoblasts when administered in an intermittent fashion to mice in vivo. Evidence is also presented herein that bovine PTH(1-34) [bPTH(1-34)] prevents glucocorticoid-induced apoptosis of osteoblastic and osteocytic cells in vitro. These observations demonstrate that the previously established anabolic effects of parathyroid hormone on the skeleton are mediated by its ability to postpone osteoblast apoptosis, as opposed to a stimulatory effect on osteoblastogenesis. Results presented herein also demonstrate that the ability of parathyroid hormone to prevent glucocorticoid-induced osteoblast and osteocyte apoptosis is due to direct interference with a private death pathway that occurs prior to activation of the final steps of apoptotic mechanism such as activation of the protease caspase-3.

The present invention is directed towards methods of screening agents for the ability to inhibit apoptosis of osteoblasts and osteocytes, thereby identifying agents capable of stimulating and/or restoring bone formation, or preventing bone loss due to treatment with agents such as glucocorticoids.

The present invention is directed to a method of reducing the number of osteoblasts undergoing apoptosis in an individual in need of such treatment, comprising the step of: administering a therapeutic dose of human parathyroid hormone [hPTH(1-34)] to said individual, wherein administration of human parathyroid hormone [hPTH(1-34)] results in a reduction in the number of osteoblasts undergoing apoptosis, thereby preventing bone loss and/or stimulating bone formation in said individual. In one aspect, the individual is osteopenic. Preferably, the individual is selected from the group consisting of an individual currently being treated with one or more glucocorticoid compounds and an individual previously treated with one or more glucocorticoid compounds. Although any route of administration of human parathyroid hormone [hPTH(1-34)] may be used, systemic, oral, intravenous, nasal spray and inhallation are preferred. Generally, the human parathyroid hormone [hPTH(1-34)] is administered in a dose of from about 10 μg/kg of body weight to about 1000 μg/kg of body weight.

The present invention is also directed to a method of screening compounds that stimulate bone formation, comprising the steps of: (a) contacting osteoblast cells with said compound; (b) determining the number of said cells undergoing apoptosis; and (c) comparing the number of apoptotic cells with osteoblast cells that have not been contacted with said compound, wherein fewer apoptotic cells following contact with said compound than in the absence of said contact indicates that said compound inhibits apoptosis resulting in stimulation of bone formation. Generally, in this method, the contacting of said osteoblast cells is selected from the group consisting of in vitro osteoblast cells and an in vivo murine animal model. Representative in vivo murine animal models are the SAMP6 mouse and the SAMR1 mouse. Generally, the stimulation of bone formation is confirmed by methods known to those having ordinary skill in this art such as measuring BMD, measuring cancellous bone area, measuring cancellous bone formation rate, measuring the number of osteoblasts per cancellous bone perimeter and measuring the number of osteocytes per bone area in said murine animal model following said contact with said compound compared with a murine animal model in the absence of said contact with said compound. The determination of apoptotic cells may be by microscopy of stained cells, TUNEL, Hoescht 33258 dye and video image analysis.

The present invention is also directed to a method of screening for compounds that decrease bone loss, comprising the steps of: (a) treating osteoblast cells with a glucocorticoid; (b) contacting said osteoblast cells with a test compound; (c) determining the number of said osteoblast cells undergoing apoptosis; and (d) comparing the number of apoptotic cells with osteoblast cells that have been treated with said glucocorticoid but were not contacted with said test compound, wherein fewer apoptotic cells following contact with said test compound than in the absence of said contact with said test compound indicates that said compound inhibits apoptosis of osteoblast cells thereby reducing bone loss. The contacting of the osteoblast cells may be in vitro osteoblast cells or in an in vivo murine animal model. Representative in vivo murine animal models isclude the SAMP6 mouse and the SAMR1 mouse. The determination of apoptotic cells may be by microscopy of stained cells, TUNEL, Hoescht 33258 dye and video image analysis.

It is specifically contemplated that pharmaceutical compositions may be prepared using the parathyroid hormone of the present invention. In such a case, the pharmaceutical composition comprises the parathyroid hormone of the present invention and a pharmaceutically acceptable carrier. A person having ordinary skill in this art would readily be able to determine, without undue experimentation, the appropriate dosages and routes of administration of this parathyroid hormone of the present invention. When used in vivo for therapy, the parathyroid hormone of the present invention is administered to the patient or an animal in therapeutically effective amounts, i.e., amounts that increase or stimulate bone formation. It will normally be administered parenterally, preferably subcutaneously by nasal spray or inhallation, but other routes of administration will be used as appropriate.

The dose and dosage regimen of the parathyroid hormone will depend upon the nature of the disease, the characteristics of the particular parathyroid hormone, e.g., its therapeutic index, the patient, the patient's history and other factors. The amount of parathyroid hormone administered will typically be in the range of about 10 to about 1000 μg/kg of patient weight. The schedule will be continued to optimize effectiveness while balanced against negative effects of treatment. See Remington's Pharmaceutical Science, 17th Ed. (1990) Mark Publishing Co., Easton, Pa.; and Goodman and Gilman's: The Pharmacological Basis of Therapeutics 8th Ed (1990) Pergamon Press; which are incorporated herein by reference. For parenteral administration, parathyroid hormone will most typically be formulated in a unit dosage injectable form (solution, suspension, emulsion) in association with a pharmaceutically acceptable parenteral vehicle. Such vehicles are preferably non-toxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. Parathyroid hormone will typically be formulated in such vehicles at concentrations of about 10 μg/ml to 1000 μg/ml.

The following examples are given for the purpose of illustrating various embodiments of the invention and are not meant to limit the present invention in any fashion:

### EXAMPLE 1

### In Vitro Effects of PTH

The number of osteoblasts, a critical determinant of bone formation and bone mass, depends both on the birth rate of these cells, which reflects the frequency of cell division of mesenchymal precursors, and on their life span, which reflects the timing of death by apoptosis. In vivo evidence indicates that intermittent administration of parathyroid hormone(1-34) increases bone formation and BMD in mice and that these changes are associated with decreased osteoblast and osteocyte apoptosis, but not with increased production of progenitors in the bone marrow.

To determine the mechanism of such actions, the effects of parathyroid hormone(1-34) on the apoptosis of cultured osteoblastic cells isolated from neonatal murine calvaria and the MLO-Y4 osteocyte cell line (provided by L. Bonewald) were examined. Chromatin condensation, nuclear fragmentation, and DNA degradation—cardinal features of apoptotic cells—were monitored by microscopic examination of cells stained with the DNA dye Hoescht 33258, or stably transfected with green fluorescent protein gene containing a nuclear localization sequence, and by DNA end labeling (TUNEL). Enumeration of apoptotic cells was performed by trypan blue staining, and correlated closely with morphologic changes and TUNEL.

In both osteoblast and osteocyte cultures, 15% of the cells were apoptotic 6 hours after addition of 100 nM dexamethasone, as compared to 4% in cultures without the steroid. This effect was completely prevented by 10 nM parathyroid hormone(1-34) or 1 mM dibutyryl-cAMP added 1 hour prior to addition of dexamethasone. The parathyroid hormone effect did not involve cytokines with anti-apoptotic properties, as neutralizing antibodies against IL-6, IL-11 or LIF did not interfere with this phenomenon. In contrast to dexamethasone-induced apoptosis, parathyroid hormone had no influence on TNF-induced apoptosis. These findings are consistent with in vivo evidence demonstrating that the anabolic effects of parathyroid hormone are due to its anti-apoptotic effects on osteoblasts and osteocytes; and that parathyroid hormone interferes directly with a private apoptosis pathway at a site(s) upstream of the induction of the degradation phase of apoptosis which is executed by caspase-3.

### EXAMPLE 2

### Calvaria and MLO-Y4 Cells

Osteoblastic calvaria cells (9) were cultured in αMEM (Gibco-BRL, Grand Island, N.Y.) supplemented with 10% FBS (Sigma Chemical Co., St. Louis, Mo.). Murine osteocyte-like MLO-Y4 cells stably transfected with EGFP were cultured on collagen coated plates in (MEM supplemented with 5% FBS and 5% bovine calf serum. Cultures were maintained for 6 hours in the presence of 10⁻⁷ M dexamethasone without or with preincubation for 1 hour with 10⁻⁸ M bPTH(1-34) and fixed in neutral buffered formalin. The pyknotic fragmented nuclei (arrows) typical of apoptotic cells were visualized with Hoescht 33258 fluorescent dye (Polysciences, Inc., Bayshore, N.Y.), used at a concentration of 1 μg/ml in 0.5 M NaCl, 10 mM Tris-HCl, 1 mM EDTA, pH 7.4) in osteoblastic calvaria cells, and by EGFP fluorescence in MLO-Y4 osteocytes.

Osteoblastic cells were isolated from calvaria of 3- to 6-day-old C57/B1 mice by sequential collagenase digestion. Cells were cultured for 5-8 days in αMEM supplemented with 10% FBS and frozen in liquid N₂ until use. MLO-Y4 cells (provided by Dr. L. Bonewald, University of Texas Health Science Center at San Antonio, San Antonio, Tex.) were transduced with the pLXSN retroviral vector containing a construct encoding enhanced green fluorescent protein (Clontech, Palo Alto, Calif.) with the SV40 large T antigen nuclear localization sequence [D. Kalderon et al., Cell 39, 499 (1984)] attached to the carboxyterminus. Stably transduced cells were selected for neomycin resistance using G418 (Sigma, St. Louis, Mo.).

### EXAMPLE 3

### Mice

4-5 month old male or female SAMR1 and SAMP6 were given daily injections of vehicle (0.9% saline, 0.01 mM β-mercaptoethanol, 0.1 mM acetic acid) or 400 ng/g body weight of hPTH(1-34) (Bachem, Torrence, Calif.) dissolved in vehicle (n=6-7 per group). Mice were fed a standard rodent diet (Agway RMH 3000, Arlington Heights, Ill.) ad libitum. The BMD of the spine and hindquarters was determined one day prior to initiation of the experiment (baseline scan) and at weekly intervals thereafter using dual-energy X-ray absorptiometry (QDR 2000, Hologic, Inc.) as described previously (3). The evaluation of each scan was based on the exact positioning and region of interest placement of the baseline scan using the “Compare” technique (4).

### EXAMPLE 4

### Experimental Methods

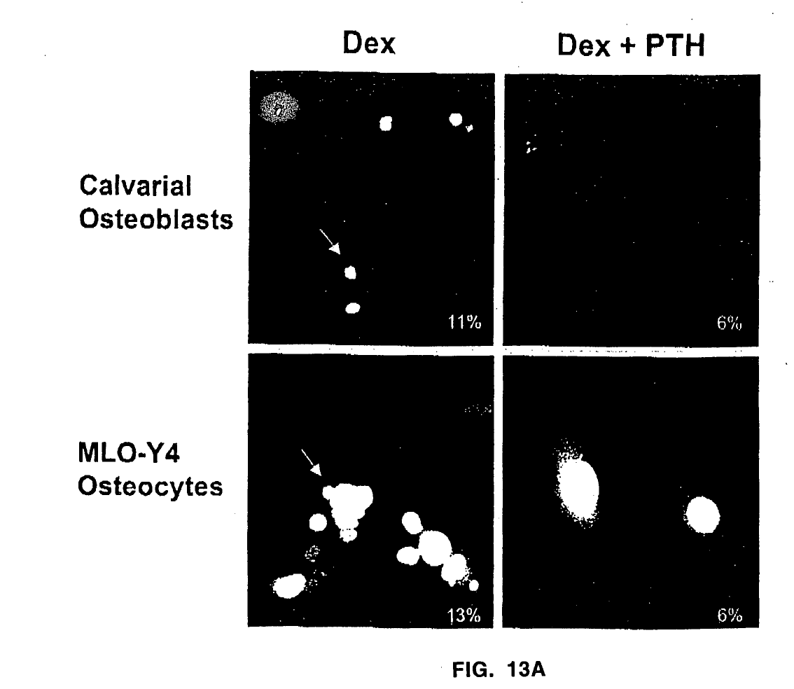

To determine the effect of PTH on osteoblast formation, function and fate, mice from the experiment shown in FIG. 11 were killed on day 28. The animals had been pretreated with tetracycline (5 μg/g, s.c.) on day 19 and 26. Osteoblast progenitors were measured using marrow cells from one femur. Cells from each animal were cultured separately at 2.5×10⁶ per 10 cm² well and maintained for 28 days in phenol red-free (MEM containing 15% preselected FBS (HyClone, Logan, Utah) and 1 mM ascorbate-2-phosphate (18). Von Kossa's method was used to visualize and enumerate colonies containing mineralized bone matrix. Because each colony is derived from a single osteoblast progenitor, the colony forming unit osteoblast (CFU-OB), the number of CFU-OB colonies reflects the number of osteoblast progenitors present in the original bone marrow isolate. The remaining femur and lumbar vertebrae were fixed in 4° C. Millonigís phosphate-buffered 10% formalin, pH 7.4 and embedded undecalcified in methyl methacrylate.

Measurements of the femoral length and the midshaft diaphyseal cortical width were made with a digital caliper at a resolution of 0.01 mm (Mitutoyo Model #500-196, Ace Tools, Ft. Smith, Ark.). Histomorphometric examination of five micron thick bone sections were performed using a computer and digitizer tablet (OsteoMetrics Inc. Version 3.00, Atlanta, Ga.) interfaced to a Zeiss Axioscope (Carl Zeiss, Inc., Thornwood, N.Y.) with a drawing tube attachment (4). Measurements were confined to the secondary spongiosa of the distal femur. The terminology and units used are those recommended by the Histomorphometry Nomenclature Committee of the American Society for Bone and Mineral Research (19). The rate of bone formation (μm²/μm/d) was calculated from the extent of bone surface labeled with tetracycline (visualized by fluorescence under UV illumination) and the distance between the labels in areas where two labels are present. Osteoid was recognized by its distinct staining characteristics and osteoblasts were identified as plump cuboidal cells on osteoid surfaces.

Apoptotic osteoblasts were detected in sections of nondecalcified vertebral bone by the TUNEL reaction (TdT-mediated dUTP nick end labeling) using reagents from Oncogene (Cambridge, Mass.) (4,7). Briefly, sections were incubated in 0.5% pepsin in 0.1 N HCl for 20 minutes at 37° C., rinsed with TBS and then incubated in 30% H2O2 in methanol for 5 minutes, and rinsed again. To improve the sensitivity of the reaction, sections were subsequently incubated for 1-2 minutes with 0.15% CuSO₄ in 0.9% NaCl (20). TUNEL-positive hypertrophic chondrocytes were observed at the bottom of the growth plates serving as an internal positive control for each bone section. The prevalence of osteoblast apoptosis was determined by inspecting 1190 and 852 osteoblasts in sections from vehicle-treated and 2514 and 1490 osteoblasts in PTH-treated SAMR1 and SAMP6 mice, respectively. For the determination of osteocyte apoptosis, 1579 and 1714 osteocytes were evaluated in vehicle-treated and 2930 and 2259 osteocytes in PTH-treated SAMR1 and SAMP6 mice, respectively. Wall width represents the amount of bone synthesized by a team of osteoblasts and was measured as the distance from the bone surface to a cement line in the underlying bone demarcating the site at which bone formation began. Osteocytes were identified inside lacunae in mineralized bone. Osteoclasts were recognized by staining with tartrate resistant acid phosphatase.

The decrease in the number of apoptotic osteoblasts in PTH-treated mice was confirmed in a second set of bone sections stained with the standard TUNEL technique (4,7), as opposed to the modified TUNEL method used for the data presented in the text and Table 1, in which CuSO₄ is added subsequent to peroxidase staining to intensify the chromogen so as to allow detection of DNA degradation as early as possible (20). Using the standard TUNEL technique, the prevalence of apoptotic osteoblasts in PTH-treated mice was 0.017% (1 out of 5,900 osteoblasts inspected in 36 sections taken from 12 mice: 6 SAMR1 and 6 SAMP6) compared to 0.37% in the vehicle-treated controls (12 out of 3,250 osteoblasts inspected in 39 sections taken from 13 mice, 6 SAMR1 and 7 SAMP6); p<0.001 vs. PTH-treated animals by logistic regression (LogXact, Cytel Corp. Cambridge, Mass.). The increase in the absolute number of TUNEL-labeled cells observed with the modified as compared to the standard technique is consistent with the contention that the former procedure allows visualization of cells undergoing the DNA degradation phase at an earlier stage. Prolongation or shortening of the time that apoptosis can be observed in a specimen as a result of using a more or less sensitive detection method must be taken into consideration when comparing estimates of the prevalence of the phenomenon in different studies (4,7). This variability of the duration of the apoptosis process in different cell types can account for reports estimating the TUNEL-labeled phase of apoptosis from as little as 1.5 to as much as 48 hours.

### EXAMPLE 5

### In Vivo Effects on Mice of bPTH

Intermittent administration of parathyroid hormone exerts an anabolic effect on the skeleton of animals and humans, most likely due to an increase in the number of osteoblasts. This number depends both on the birth rate (reflecting the frequency of division of mesenchymal progenitors) and the life span (reflecting the timing of apoptosis). The present invention examines the effects of intermittent parathyroid hormone administration in mice with either normal (SAMR1) or defective (SAMP6) osteoblastogenesis at four months of age, a time at which both strains have achieved peak bone mass.

Mice (6-7 per group) were given daily subcutaneous injections of 400 ng bovine parathyroid hormone (1-34) per gram of body weight or vehicle for a period of 4 weeks. BMD was monitored weekly by DEXA. One femur was used for tetracycline based dynamic histomorphometry and the other for determination of osteoblast progenitors in ex vivo bone marrow cultures. Spine and hindquarter BMD increased gradually in parathyroid hormone-treated mice of either strain reaching 4% and 15%, respectively, over the pretreatment values by 4 weeks. Parathyroid hormone also increased cancellous bone area and bone formation rate (2-3 fold), as well as the number of osteoblasts per cancellous bone perimeter and the number of osteocytes per bone area (2-fold) in both strains.

These changes could not be accounted for by hypertrophy of lining cells and their subsequent conversion to osteocytes because the increased osteocyte density in the expanded cancellous bone area was much too great. Unlike the increase in mature cell numbers, the number of CFU-OB formed per 10⁶ marrow cells in ex vivo bone marrow cultures did not change in either strain (SAMR1: vehicle=34±7, PTH=34±9; SAMP6: vehicle=17±2, PTH=21±5). Parathyroid hormone did, however, decrease osteoblast apoptosis.

As detected by DNA end-labeling (TUNEL), 0.33% and 0.40% of osteoblasts were apoptotic in bone of SAMR1 and SAMP6 mice, respectively (N=3250), whereas only 0.02% of the 4200 osteoblasts examined in parathyroid hormone-treated mice were apoptotic (P<0.02 by z-test). Based on an osteoblast lifetime of 300 hours, and a 3 hour duration of apoptosis, it was calculated that parathyroid hormone postponed apoptosis of the 30-40% of osteoblasts that would normally undergo this process during bone remodeling. These findings indicate that suppression of osteoblast apoptosis, rather than increased osteoblastogenesis, is the mechanism by which intermittent administration of parathyroid hormone stimulates bone formation. This effect is sufficient to account for the increase in the number of osteoblast and osteocytes and, thereby, the anabolic effect of the hormone.

### EXAMPLE 6

### In Vivo Effects of hPTH on Mice

Daily subcutaneous injections of hPTH (1-34) over a four week period progressively increased bone mineral density (BMD) in adult mice with normal bone mass (SAMR1) or in mice with osteopenia (3) due to impaired osteoblastogenesis and decreased bone formation (SAMP6) (FIG. 11A). This effect was greater in the hindlimbs than in the spine and did not involve bone growth as reflected by the lack of change in the length of the femur or its width at the diaphysis (Table 1). Remarkably, the increase in BMD was similar in the two strains, even though the baseline values were different (FIG. 11B).

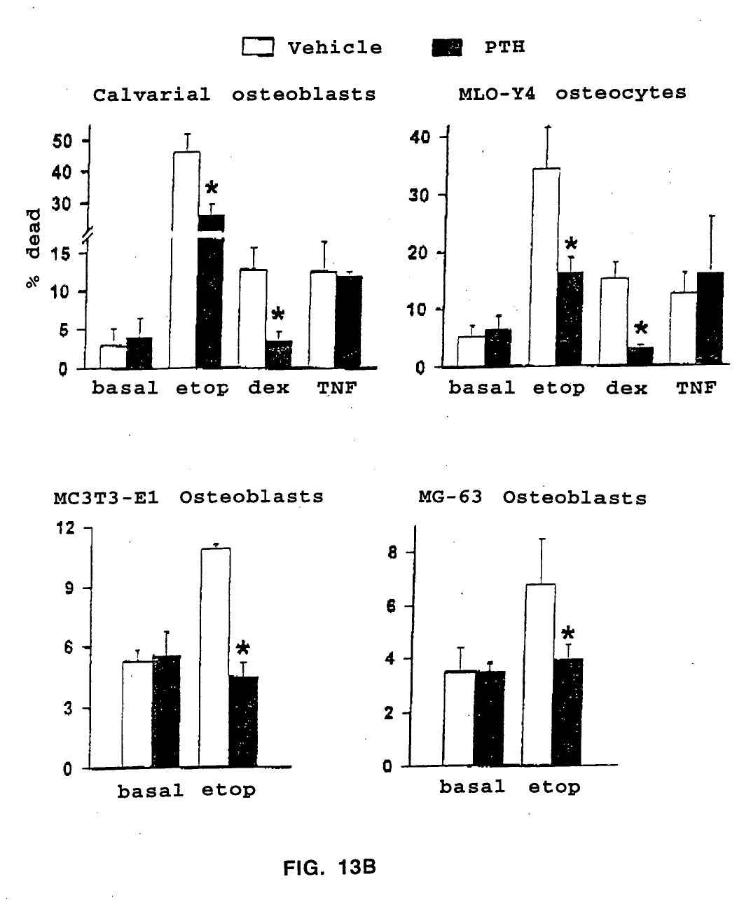

Consistent with the BMD increase, histomorphometric analysis of sections of cancellous bone from the distal femurs of mice of either strain treated with PTH showed increased cancellous bone area that correlated with the increase in hindlimb BMD (r=0.53, P<0.001). The latter could be explained by the increase in the number of osteoblasts covering the bone surface and by the increased rate of bone formation (Table 1). As expected from these changes, the amount of osteoid, the matrix produced by osteoblasts, was also increased, as was the amount of bone estimated to be produced by each team of osteoblasts (wall width). The number of osteoclasts on the cancellous bone surface was not changed with PTH treatment, indicating that bone resorption and turnover were not affected by the hormone. The newly formed bone had normal lamellar architecture as opposed to woven bone seen in severe hyperparathyroidism (FIG. 12).

As osteoblasts are short-lived cells (approximately 200 hours in mice) (4,7), the increase in the number of osteoblasts seen in the PTH-treated mice could result from either an increase in the formation of new osteoblasts or the prolongation of their life span. Enumeration of osteoblast progenitors in ex vivo bone marrow cell cultures, a reliable index of de novo osteoblastogenesis (3), showed no difference between PTH and vehicle-treated animals (Table 1). The lack of effect of PTH on osteoblast progenitors was documented in the normal mouse strain (SAMR1) as well as in the strain with the diminished baseline osteoblastogenesis (SAMP6), clearly demonstrating that an increase in the formation of new osteoblasts could not account for the increased osteoblast numbers seen in both strains.

Strikingly, however, the percentage of osteoblasts undergoing apoptosis, as determined by TUNEL-labeling, was greatly decreased in animals of either strain receiving PTH (Table 1). Specifically, the average number of apoptotic osteoblasts, determined separately for each animal, was 0.4±0.5% (range 0-1.4%, n=6) for PTH-treated SAMR1 as compared to 1.7±1.0% (range 0.5-3.2%, n=6) for vehicle-treated SAMR1, p<0.05. Similarly, the average number of apoptotic osteoblasts in PTH-treated SAMP6 was 0.1±0.3% (range 0-0.8%, n=6) as compared to 2.2±1.4% (range 0.9-4.7%, n=7) for vehicle-treated SAMP6, p<0.05 (8).

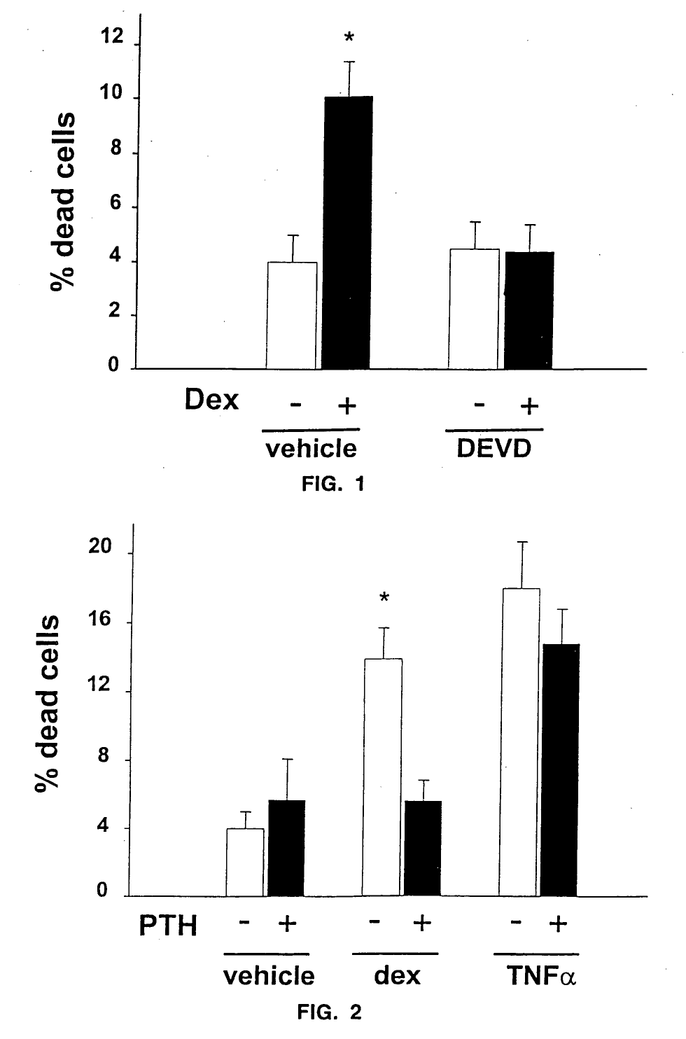

Table 1 shows the effect of PTH on osteoblast formation, function and fate. Mice from the experiment shown in FIG. 1 were killed on day 28. The animals had been pretreated with tetracycline (5 μg/g, s.c.) on day 19 and 26. Osteoblast progenitors were measured using marrow cells from one femur. The average number of nucleated cells obtained from the femur of PTH treated animals (19.8±3.2×10⁶ from SAMR1; 24.3±3.0×10⁶ from SAMP6) was indistinguishable from animals receiving vehicle (21.0±2.7×10⁶ from SAMR1; 21.5±3.2×10⁶ from SAMP6). The remaining femur and lumbar vertebrae were fixed and embedded undecalcified in methylmethacrylate (3,4,15). Femurs were used for histomorphometric analysis and vertebrae were used for apoptosis determinations. Because osteoblasts in remodeling bone comprise a team, they were identified as cuboidal cells in a row of at least three, lining the osteoid-covered trabecular perimeter. Osteocytes were identified inside lacunae of mineralized cancellous bone. For detection of apoptotic cells, sections were incubated with CuSO₄ to enhance staining of the peroxidase reaction production during the TUNEL procedure, as described in Methods. Osteoblasts and osteocytes exhibiting both brown staining due to TUNEL and the morphological feature of nuclear condensation were counted as apoptotic. With these precautions, TUNEL has been unequivocally associated with apoptosis of osteoblasts and osteocytes in bone (4,29,50). See Table 2 for a summary of apoptotic and non-apoptotic cell counts in individual animals. The data shown represent the mean (±s.d.) of each measurement determined from bones from each animal. ^{A}P<0.05 vs. vehicle by one-tailed Students t-test. ^{B}Two-way ANOVA was used to detect overall effects of PTH. ^{C}P<0.001 vs. vehicle by GSK categorical general linear regression (26).

Table 2 shows the effect of PTH on apoptosis of osteoblasts and osteocytes in vertebral cancellous bone. Osteoblasts (OB) and osteocytes (OCT) were identified in sections of lumbar vertebrae, and those exhibiting both brown staining due to TUNEL and pyknotic nuclei were counted as apoptotic as described in Table 1. Results are from two separate TUNEL staining procedures. In the first, TUNEL was performed without CuSO₄ enhancement (“w/o Cu”), and data were pooled from animals from each group for statistical analysis because of the low number of apoptotic osteoblasts visualized with this method. In the second, CuSO₄ (“with Cu”) was used to enhance TUNEL staining, and counts from each animal are shown. With the exception of the apoptotic osteocyte counts in the vehicle treated SAMP6 group, there were no significant differences among the animals within each group. Pooled data (“w/o Cu”) were analyzed by logistic regression. ^{A}P<0.0001 vs. vehicle. Data from individual animals were analyzed by GSK. ^{B}P<0.001 vs. vehicle.

TABLE 1

| | SAMR1 | | SAMP6 | | Overall Effect |
|---|---|---|---|---|---|
| | vehicle | PTH | vehicle | PTH | of PTH^{B} |
| CFU-OB | 34 ± 7 | 34 ± 9 | 17 ± 3 | 21 ± 5 | NS |
| (# per 10⁶ marrow cells) | | | | | |
| Osteoblast perimeter (%) | 7.1 ± 2.9 | 11.8 ± 6.8^{A} | 5.7 ± 3.5 | 13.2 ± 4.9^{A} | P < 0.05 |
| Apoptotic osteoblasts (%) | 1.7 ± 1.0 | 0.4 ± 0.5^{C} | 2.2 ± 1.4 | 0.1 ± 0.3^{B,C} | P < 0.05 |
| Bone area (% of tissue area) | 9.0 ± 4.2 | 23.2 ± 11.3^{A} | 8.9 ± 2.9 | 12.6 ± 3.3^{A} | P < 0.05 |
| Bone formation rate (μm/μm²/d) | 0.087 ± 0.039 | 0.361 ± 0.413^{A} | 0.071 ± 0.010 | 0.172 ± 0.133 | P < 0.05 |
| Mineralizing perimeter (%) | 8.92 ± 4.67 | 14.96 ± 4.82^{A} | 6.56 ± 3.24 | 9.01 ± 5.07 | P < 0.05 |
| Mineral apposition rate (μm/d) | 1.09 ± 0.43 | 1.14 ± 0.87 | 1.07 ± 0.23 | 1.77 ± 0.51 | NS |
| Trabecular width (μm) | 37.2 ± 11.0 | 56.7 ± 21.2^{A} | 39.9 ± 9.1 | 51.2 ± 8.5^{A} | P < 0.05 |
| Wall width (_m) | 10.2 ± 2.5 | 13.4 ± 3.2^{A} | 5.4 ± 0.9 | 8.2 ± 2.3^{A} | P < 0.05 |
| Osteoid perimeter (%) | 13.2 ± 7.8 | 17.4 ± 4.9 | 9.9 ± 5.5 | 26.2 ± 10.4^{A} | P < 0.05 |
| Osteocyte areal density | 8.2 ± 3.1 | 14.8 ± 6.4^{A} | 14.3 ± 5.7 | 20.1 ± 7.2^{A} | P < 0.05 |
| (# per bone area) | | | | | |
| Apoptotic osteocytes (%) | 1.7 ± 0.5 | 0.2 ± 0.2^{C} | 2.5 ± 2.0 | 0.4 ± 0.3^{C} | P < 0.05 |
| Osteoclast perimeter (%) | 1.3 ± 0.8 | 0.7 ± 0.8 | 2.3 ± 2.5 | 1.0 ± 1.1 | NS |
| Femoral length (mm) | 15.82 ± 0.51 | 16.14 ± 0.48 | 15.03 ± 0.66 | 15.34 ± 0.37 | NS |
| Diaphyseal cortical width (mm) | 0.59 ± 0.38 | 0.54 ± 0.13 | 0.56 ± 0.13 | 0.59 ± 0.10 | NS |

TABLE 2

| | Sample, | | | % | | Total | % |
|---|---|---|---|---|---|---|---|
| | or Mouse | Apoptotic | Total OB | Apoptotic | Apoptotic | OTC | Apoptotic |
| Group | ID | OB | Counted | OB | OTC | Counted | OCT |
| SAMR1, | Pooled | 5 | 1500 | 0.3 | — | — | — |
| Veh | (w/o Cu) | | | | | | |
| | 5-6 | 5 | 271 | 1.8 | 7 | 368 | 1.9 |
| | (with Cu) | | | | | | |
| | 5-15 | 9 | 280 | 3.2 | 9 | 350 | 2.5 |
| | 5-17 | 6 | 258 | 2.3 | 4 | 240 | 1.6 |
| | 5-20 | 1 | 199 | 0.5 | 2 | 237 | 0.8 |
| | 5-14 | 1 | 113 | 0.9 | 4 | 244 | 1.6 |
| | 5-1 | 1 | 69 | 1.4 | 2 | 98 | 1.8 |
| Mean ± | | | | 1.7 ± 1.0 | | | 1.7 ± 0.5 |
| s.d: | | | | | | | |
| SAMR1, | Pooled | 0 | 3700 | 0^{A} | — | — | — |
| PTH | (w/o Cu) | | | | | | |
| | 5-7 | 0 | 206 | 0 | 0 | 403 | 0 |
| | (with Cu) | | | | | | |
| | 5-8 | 1 | 328 | 0.3 | 1 | 477 | 0.3 |
| | 5-18 | 1 | 372 | 0.3 | 2 | 489 | 0.3 |
| | 5-2 | 3 | 206 | 1.4 | 2 | 618 | 1.4 |
| | 5-9 | 2 | 397 | 0.5 | 1 | 669 | 0.5 |
| | 5-11 | 0 | 151 | 0 | 0 | 262 | 0 |
| Mean ± | | | | 0.4 ± 0.5^{B} | | | 0.2 ± 0.2^{B} |
| s.d: | | | | | | | |
| SAMP6, | Pooled | 7 | 1750 | 0.4 | — | — | — |
| Veh | (w/o Cu) | | | | | | |
| | 7-19 | 6 | 180 | 3.4 | 9 | 204 | 4.3 |
| | (with Cu) | | | | | | |
| | 7-11 | 3 | 64 | 4.7 | 4 | 302 | 1.3 |
| | 7-6 | 1 | 114 | 0.9 | 10 | 169 | 5.7 |
| | 7-12 | 3 | 185 | 1.6 | 1 | 353 | 0.3 |
| | 7-17 | 2 | 109 | 1.8 | 9 | 342 | 2.6 |
| | 7-21 | 1 | 88 | 1.1 | 1 | 208 | 0.5 |
| | 7-24 | 2 | 113 | 1.8 | 11 | 384 | 2.8 |
| Mean ± | | | | 2.2 ± 1.4 | | | 2.5 ± 2.0 |
| s.d: | | | | | | | |
| SAMP6, | Pooled | 1 | 2200 | 0.05^{A} | — | — | — |
| PTH | (w/o Cu) | | | | | | |
| | 7-1 | 0 | 289 | 0 | 3 | 500 | 0.6 |
| | (with Cu) | | | | | | |
| | 7-4 | 0 | 387 | 0 | 1 | 487 | 0.2 |
| | 7-7 | 3 | 400 | 0.8 | 2 | 350 | 0.7 |
| | 7-9 | 0 | 160 | 0 | 2 | 321 | 0.6 |
| | 7-16 | 0 | 120 | 0 | 1 | 293 | 0.3 |
| | 7-23 | 0 | 134 | 0 | 0 | 303 | 0 |
| Mean ± | | | | 0.1 ± 0.3^{B} | | | 0.4 ± 0.3^{B} |
| s.d: | | | | | | | |

### EXAMPLE 7

### Effect of PTH on Osteocytes

PTH-treated mice also exhibited increased osteocyte density—i.e. number per cancellous bone area (Table 1 and FIG. 12). Osteocytes are former osteoblasts that have completed their bone-forming function and are encased within lacunae of the mineralized bone matrix, one of the three possible fates of matrix synthesizing cells, the other two being apoptosis and conversion to lining cells. Hence, an increase in osteocyte density is consistent with, and can only be accounted for by, a suppression of osteoblast apoptosis. Besides the effect on osteoblast apoptosis, intermittent PTH administration also inhibited osteocyte apoptosis (Table 1).

### EXAMPLE 8

### Anti-Apoptotic Effect of PTH

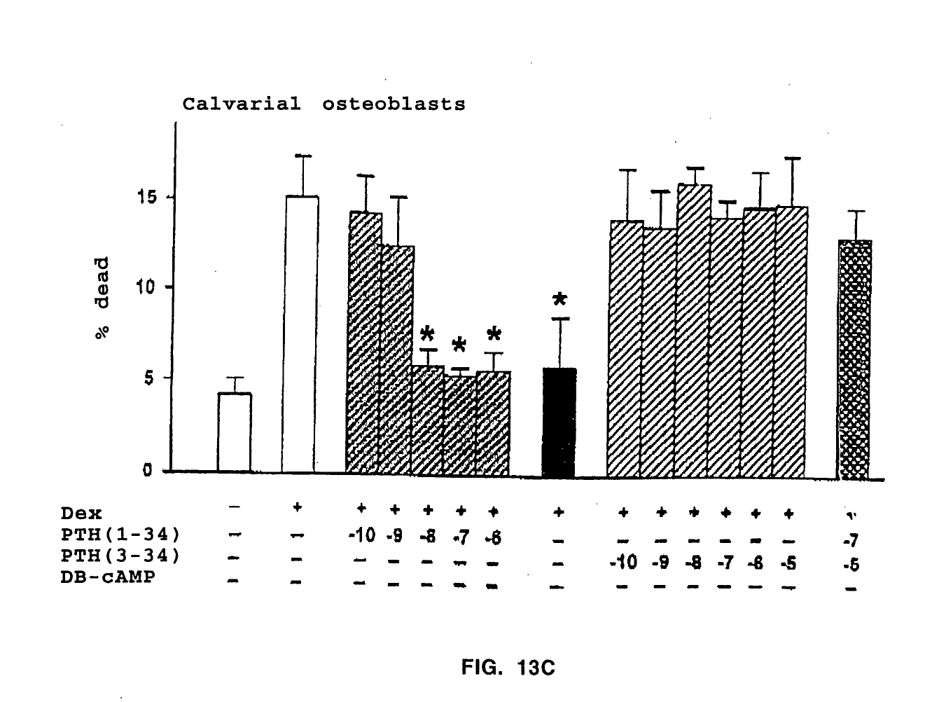

To determine whether the anti-apoptotic effect of PTH was due to direct action of the hormone on osteoblasts and osteocytes, as opposed to indirect actions mediated by compensatory changes, the effect of PTH on apoptosis was examined using cell cultures. Induction of apoptosis by either etoposide or dexamethasone was attenuated by PTH in primary cultures of osteoblasts isolated from neonatal murine calvaria (9) whereas induction of apoptosis by TNF was not (FIG. 13A,B). These findings were reproduced using murine osteoblastic MC3T3-E1 cells as well as human osteoblastic MG-63 cells (10). The anti-apoptotic effect of PTH could be blocked by the PTH/PTHrP receptor antagonist bPTH(3-34) and was mimicked by dibutyryl-cAMP, indicating that it was mediated through the PTH/PTHrP receptor and subsequent activation of adenylate cyclase (FIG. 13C). PTH also prevented etoposide- and dexamethasone-induced apoptosis, but not TNF-induced apoptosis in an established murine osteocyte-like cell line, MLO-Y4 (11), stably transfected with the enhanced green fluorescent protein (EGFP) gene containing a nuclear localization sequence, (FIG. 13A,B).

### SUMMARY

The data presented herein demonstrates that intermittent administration of PTH stimulates bone formation, not by increasing the proliferation of osteoblast precursors but by preventing osteoblast apoptosis—the fate of the majority of these cells under normal conditions (4, 7)—thereby prolonging the time spent in performing their matrix synthesizing function. The anti-apoptotic effect of PTH is exerted directly on osteoblasts, requires binding of the hormone to the PTH/PTHrP receptor, is mediated by cAMP-generated signals that interfere with some but not all death pathways, and occurs upstream of the common executing phase of apoptosis. Consistent with the results of the present studies in mice, the ability of intermittent administration of PTH to increase osteoblast numbers was not accompanied by an increase in the replication of osteoblast progenitors in the rat (12). Moreover, the demonstration of the ability of PTH to inhibit osteoblast apoptosis is in full agreement with the anti-apoptotic effect of PTHrP on chondrocytes during endochondral bone development (13).

Results of a clinical study have shown that daily subcutaneous injections of PTH is an effective treatment for glucocorticoid-induced osteoporosis (15). The decreased bone formation rate and wall thickness of trabeculae, indicators of diminished work by osteoblasts and the in situ death of portions of bone that characterize glucocorticoid-induced osteoporosis can be accounted for by a suppressive effect of glucocorticoids on osteoblastogenesis and promotion of apoptosis of osteoblasts and osteocytes (4). The elucidation of the anti-apoptotic effects of PTH in vivo along with the evidence that PTH antagonizes the pro-apoptotic effects of glucocorticoids in vitro are in full agreement with the published clinical observations. Furthermore, they provide both a mechanistic explanation for the efficacy of PTH in glucocorticoid-induced osteoporosis as well as compelling evidence that its anti-apoptotic properties make PTH a rational pharmacotherapeutic choice for this condition.

Apart from decreased cell death, another potential source of new osteoblasts is the lining cells that cover quiescent bone surfaces (16). These cells were once matrix synthesizing osteoblasts, and have escaped apoptosis or encasement within bone as osteocytes to remain on the bone surface. It has been suggested that PTH can stimulate lining cells to undergo hypertrophy and to resume matrix synthesis (12,17). Such hypertrophy was not observed, but a contribution from this mechanism cannot be excluded from the results of the present report. Nevertheless, the magnitude of the anti-apoptotic effect of PTH makes it unnecessary to invoke another explanation for increased bone formation.

In conclusion, the data presented herein demonstrate that prevention of osteoblast apoptosis is the principal mechanism for the anabolic effects of PTH on bone. Increasing the work-output of a cell population by suppressing apoptosis represents a novel biologic paradigm for regenerating tissues in general and a rational pharmacotherapeutic strategy for rebuilding bone in particular. PTH, and possibly PTH mimetics and non-peptide inhibitors of private apoptotic pathways in osteoblasts, should provide much needed therapies for osteopenias, in particular those in which osteoblast progenitors are low, such as age-related and glucocorticoid induced osteoporosis.

The following references were cited herein:
1. S. C. Manolagas & R. L. Jilka, N. Engl. J. Med. 332, 305 (1995).
2. R. L. Jilka et al., Science 257, 88 (1992).
3. R. L. Jilka et al., J. Clin. Invest. 97, 1732 (1996).
4. R. S. Weinstein et al., ibid., 102, 274 (1998).
5. D. W. Dempster et al., Endocr. Rev. 14, 690 (1993); J. Reeve et al., Lancet 1(7968), 1035 (1976); J. Reeve et al., Br. Med. J. 280, 1340 (1980); C. S. Tam et al., Endocrinology 110, 506 (1982); J. S. Finkelstein et al., JAMA 280, 1067 (1998).
6. A. F. Stewart, Bone 19, 303 (1996); B. H. Vickery et al., J. Bone Miner. Res. 11, 1943 (1996).
7. R. L. Jilka et al., J. Bone Miner. Res. 13, 793 (1998).
8. W. Bursch et al., Carcinogenesis 11, 847 (1990); M. Pompeiano et al., Cell Death Differ. 5, 702 (1998).
9. R. L. Jilka, Bone. 7, 29 (1986).
10. H. Sudo et al., J. Cell Biol. 96, 191 (1983); R. T. Franceschi et al., J. Biol. Chem. 263, 18938 (1988).
11. Y. Kato et al., J. Bone Miner. Res. 12, 2014 (1997).
12. H. Dobnig & R. T. Turner, Endocrinology 136, 3632 (1995).
13. M. Amling et al, J. Cell Biol. 136, 205 (1997); K. Lee et al., Endocrinology 137, 5109 (1996).
14. E. M. Aarden et al., J. Cellular Biochem. 55, 287 (1994).
15. N. E. Lane et al., J. Clin. Invest. 102, 1633 (1998).
16. S. C. Miller & W. S. S. Jee, Bone. Volume 4: Bone Metabolism and Mineralization, B. K. Hall, Ed. (CRC Press, Boca Raton, 1992), p. 1.
17. D. Leaffer et al., Endocrinology 136, 3624 (1995).
18. R. L. Jilka et al., J. Clin. Invest. 101, 1942 (1998).
19. A. M. Parfitt et al., J. Bone Miner. Res. 2, 595 (1987).
20. S. Hsu & E. Soban, J. Histochem. Cytochem. 30, 1079 (1982); B. G. Short et al., ibid, 45, 1299 (1997).
21. S. C. Wright et al., J. Exp. Med. 186, 1107 (1997); N. A. Thornberry & Y. Lazebnik, Science 281, 1312 (1998).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A method of increasing the lifespan of osteoblasts in a bone-containing host in need of preventing bone loss or stimulating bone formation, wherein the host is currently being treated with one or more glucocorticoid compounds or the host is experiencing adverse bone effects resulting from contact with one or more glucocorticoid compounds, comprising the step of:
administering therapeutically effective dosages of at least 10 micrograms per kilogram body weight of isolated parathyroid fragment wherein said parathyroid fragment is parathyroid hormone (1-34) to said host.

2. The method of claim 1, wherein said administration is systemic, oral, intravenous, nasal spray or inhalation.

3. The method of claim 1, wherein said host is a human.
